(19) United States
(12) Patent Application Publication
(10) Pub. No.: US 2026/0248615 A1
(43) Pub. Date: Aug. 27, 2026

(54) Cardiac Reconstruction Implant

(71) Applicant: GUANGDONG PULSE MEDICAL TECHNOLOGY CO., LTD., Zhuhai, Guangdong (CN)

(72) Inventors: Chengmin RUAN, Zhuhai, Guangdong (CN); Yalun JIANG, Zhuhai, Guangdong (CN); Zhenhua HAO, Zhuhai, Guangdong (CN); Yulin LIANG, Zhuhai, Guangdong (CN); Wentao XIE, Zhuhai, Guangdong (CN); Yanhong SUN, Zhuhai, Guangdong (CN)

(73) Assignee: GUANGDONG PULSE MEDICAL TECHNOLOGY CO., LTD., Zhuhai, Guangdong (CN)

(21) Appl. No.: 18/992,496

(22) PCT Filed: Oct. 28, 2022
(86) PCT No.: PCT/CN2022/128132

§ 371 Date: Jan. 8, 2025

(30) Foreign Application Priority Data
Jul. 8, 2022 (CN) 202210805770.8

(51) Int. Cl. A61F 2/24

(52) U.S. Cl. CPC A61F 2/2487; A61F 2002/249; A61F 2220/0008

(57) ABSTRACT

A cardiac reconstruction implant includes an isolation body and a contractile outer mesh; the isolation body and the contractile outer mesh are integrally formed by weaving; the isolation body is released in a cardiac ventricle of a heart, and the woven isolation body isolates an ineffective cardiac chamber. Balloon expansion is no longer required during an implantation of the woven isolation body, and a force applied to the isolation body is dispersed. The contractile outer mesh covers a surface of the heart, the contractile outer mesh is able to provide a contractile force to an exterior of the heart.

### CROSS-REFERENCE TO RELATED APPLICATION

The disclosure is a National Stage Filing of the PCT International Application No: PCT/CN2022/128132 filed on Oct. 28, 2022, which claims the priority to Chinese Patent Application No. CN202210805770.8, filed with the China National Intellectual Property Administration (CNIPA) on Jul. 8, 2022, and entitled “Cardiac Reconstruction Implant”, which is hereby incorporated by reference in its entirety.

### TECHNICAL FIELD

The disclosure relates to the technical field of medical devices, and in particularly to a cardiac reconstruction implant.

### BACKGROUND

Heart failure is the ultimate fate of most cardiovascular diseases and can lead to death, and the heart of a patient cannot provide sufficient blood flow to meet the requirements of the body. Symptoms of heart failure negatively impact the quality of life and include shortness of breath, persistent coughing or wheezing, excessive fluid buildup (oedema) in body tissues, fatigue, loss of appetite or nausea, mental sluggishness, and tachycardia. In recent years, the incidence of heart failure has increased annually and has become a serious public health problem. After a heart attack, many patients experience left ventricular enlargement, leading to a decreased cardiac output, which in turn causes symptoms of heart failure, such as shortness of breath. A left ventricular volume reduction device is a catheter-based minimally invasive treatment technology for isolating damaged myocardium. It separates a non-functional part of the heart from a healthy and functional part, thereby reducing the total volume of the left cardiac ventricle and restoring the morphology and function thereof.

The left ventricular volume reduction device in the related art has the following drawbacks: since an elastic nickel-titanium support skeleton is used for supporting, balloon expansion is needed during implantation to fully expand an umbrella body, thereby increasing a certain surgical operation time and risk; after implantation, with ventricular motion, an instrument skeleton structure experiences concentrated stress and is prone to fracture; a membrane covering the skeleton structure tends to contract and fold along with ventricular motion, leading to poor sealing performance and a poor volume reduction effect; long-term use may lead to tearing; in addition, for left ventricular volume reduction devices in the related art, the left ventricular volume reduction device placed within a cardiac ventricle to reduce the ventricular volume, but they do not provide contraction during a systolic phase, and therefore, the problem of insufficient contractile force during ventricular expansion still persists.

### SUMMARY

Some embodiments of the disclosure provide a cardiac reconstruction implant, including: an isolation body and a contractile outer mesh;
the isolation body and the contractile outer mesh are integrally formed by weaving, and the isolation body is released in a cardiac ventricle of a heart to isolate an ineffective cardiac chamber; and the contractile outer mesh covers a surface of the heart to provide a contractile force to an exterior of the heart.

In some embodiments, the cardiac reconstruction implant further includes a fixation waist;
the isolation body and the contractile outer mesh are integrally woven and connected by the fixation waist; the fixation waist is fixed at a cardiac apex of the heart, so that the contractile outer mesh spreads and covers the surface of the heart with the fixation waist as a center.

In some embodiments, the isolation body, the fixation waist and the contractile outer mesh are sequentially formed by weaving an elastic filament, and the contractile outer mesh covers an exterior of the isolation body.

In some embodiments, the isolation body includes an expansion segment and an extension segment;
the expansion segment is connected to the fixation waist by the extension segment; the expansion segment is located at an end, away from the cardiac apex, of the heart; the expansion segment is configured to conform to a ventricle wall; and the extension segment is able to conform to the ventricle wall from the expansion segment to the cardiac apex.

In some embodiments, a diameter of the expansion segment is greater than a diameter of the extension segment, and the extension segment fully conforms to a shape of the heart.

In some embodiments, the contractile outer mesh is arcuately expanded along the fixation waist to completely cover the exterior of the heart.

In some embodiments, the isolation body is formed as a double-layer woven mesh, and an occlusive membrane is fixed inside the double-layer woven mesh of the isolation body; and the contractile outer mesh is formed as a single-layer or double-layer woven mesh.

In some embodiments, the isolation body is formed as a double-layer woven mesh, and an occlusive membrane is fixed on a surface of the double-layer woven mesh of the isolation body; and the contractile outer mesh is formed as the single-layer or double-layer woven mesh.

In some embodiments, the isolation body is formed as a single-layer woven mesh, and an occlusive membrane is fixed on a surface of the single-layer woven mesh or an inner cavity of the isolation body; and the contractile outer mesh is formed as the single-layer woven mesh.

In some embodiments, the contractile outer mesh is formed as a double-layer woven mesh, and an occlusive membrane is fixed on the contractile outer mesh; and
the isolation body is formed as a single-layer or double-layer woven mesh, and the occlusive membrane is fixed on the isolation body.

Some other embodiments of the disclosure further provide a cardiac reconstruction implant, including: an isolation body and a contractile outer mesh;
the isolation body and the contractile outer mesh are fixedly connected, and the isolation body is released in a cardiac ventricle to isolate an ineffective cardiac chamber of a heart; and the contractile outer mesh covers a surface of the heart to provide a contractile force to an exterior of the heart.

In some embodiments, the cardiac reconstruction implant further includes a fixation waist;
the isolation body and the contractile outer mesh are fixedly connected by the fixation waist; the fixation waist is fixed to a cardiac apex of the heart, so that the contractile outer mesh spreads and covers the surface of the heart with the fixation waist as a center.

In some embodiments, the isolation body, the fixation waist and the contractile outer mesh are prepared and formed by an elastic material, and the contractile outer mesh covers an exterior of the isolation body.

In some embodiments, the isolation body includes an expansion segment and an extension segment;
the expansion segment is connected to the fixation waist by the extension segment; the expansion segment is located at an end, away from the cardiac apex, of the heart; the expansion segment is configured to conform to a ventricle wall; and the extension segment is able to conform to the ventricle wall from the expansion segment to the cardiac apex.

In some embodiments, a diameter of the expansion segment is greater than a diameter of the extension segment, and the extension segment fully conforms to a shape of the heart.

In some embodiments, the contractile outer mesh is arcuately expanded along the fixation waist to completely cover the exterior of the heart.

In some embodiments, the isolation body is a double-layer mesh structure, and an occlusive membrane is fixed to the double-layer mesh structure of the isolation body; and the contractile outer mesh is a single-layer or double-layer mesh structure.

In some embodiments, the contractile outer mesh is a double-layer mesh structure, and an occlusive membrane is fixed on the contractile outer mesh; and
the isolation body is a single-layer or double-layer mesh structure, and an occlusive membrane is fixed on the isolation body.

In some embodiments, the contractile outer mesh is a single-layer mesh structure, and an occlusive membrane is fixed on the contractile outer mesh; and the isolation body is a single-layer mesh structure, and the occlusive membrane is fixed on the isolation body.

In some embodiments, the cardiac reconstruction implant further includes a connector;
the connector is connected to the fixation waist, and the connector is configured to connect to an external delivery system to contract the isolation body, the fixation waist and the contractile outer mesh into the external delivery system.

In some embodiments, a woven layer of the isolation body in contact with a ventricle wall is provided with at least one fixation anchor, and the fixation anchor extends into the ventricle wall along the woven layer of the isolation body.

In some embodiments, a mesh structure layer of the isolation body in contact with a ventricle wall is provided with at least one fixation anchor, and the fixation anchor extends into the ventricle wall along the mesh structure layer of the isolation body.

In some embodiments, the fixation waist is provided with one or more fixation anchors, and the fixation anchors extend along the fixation waist to the cardiac reconstruction implant and penetrate into a myocardial inner wall of the cardiac apex.

The disclosure further provides a cardiac reconstruction method, including: implanting the cardiac reconstruction implant according to any one of the above mentioned into the heart through a cardiac apex intervention or percutaneous intervention.

### BRIEF DESCRIPTION OF THE DRAWINGS

In order to explain the embodiments of the disclosure or the technical solutions in the related art more clearly, the accompanying drawings required by the embodiments or descriptions in the related art are introduced briefly below. Obviously, the accompanying drawings in the description below are merely some embodiments of the disclosure. Those of ordinary skill in the art can also obtain other accompanying drawings based on the provided accompanying drawings without paying creative work.

FIG. 1 is an overall structural schematic diagram of a cardiac reconstruction implant according to an embodiment of the disclosure;

FIG. 2 is a partially enlarged structural schematic diagram of a cardiac reconstruction implant according to an embodiment of the disclosure;

FIG. 3 is a structural schematic diagram of an isolation body of a cardiac reconstruction implant formed as a double-layer woven mesh according to an embodiment of the disclosure;

FIG. 4 is an overall structural schematic diagram of a cardiac reconstruction implant according to an embodiment of the disclosure;

FIG. 5 is a schematic diagram of a preliminary process of a cardiac reconstruction implant being released into a left ventricle through percutaneous intervention and covering an exterior of a heart according to an embodiment of the disclosure;

FIG. 6 is a schematic diagram of an intermediate process of the cardiac reconstruction implant being released into a left ventricle through percutaneous intervention and covering an exterior of a heart according to an embodiment of the disclosure;

FIG. 7 is a schematic diagram of a completed process of the cardiac reconstruction implant being released into a left ventricle through percutaneous intervention and covering an exterior of a heart according to an embodiment of the disclosure;

FIG. 8 is a schematic diagram of a preliminary process of the cardiac reconstruction implant being released into the left ventricle through a cardiac apex and covering an exterior of a heart according to an embodiment of the disclosure;

FIG. 9 is a schematic diagram of an intermediate process of the cardiac reconstruction implant being released into the left ventricle through the cardiac apex and covering an exterior of a heart according to an embodiment of the disclosure;

FIG. 10 is a schematic diagram of a completed process of the cardiac reconstruction implant being released into the left ventricle through the cardiac apex and covering an exterior of a heart according to an embodiment of the disclosure;

FIG. 11 is a state diagram of a cardiac reconstruction implant being released into a left ventricle and covering an exterior of a heart according to an embodiment of the disclosure.

Reference signs: 100—Isolation body; 101—Expansion segment; 102—Extension segment; 200—Contractile outer mesh; 300—Fixation waist; 400—Connector; 500—Delivery system; 600—Fixation anchor.

### DETAILED DESCRIPTION OF EMBODIMENTS

The technical solutions of the disclosure will be clearly and completely described below in conjunction with the embodiments. Obviously, the described embodiments are only a part of the embodiments of the disclosure, but not all of the embodiments. All other embodiments obtained by those of ordinary skill in the art based on the embodiments in the disclosure without creative efforts shall fall within the protection scope of the disclosure.

It should be noted that: Similar reference numbers and letters in the following drawings represent similar items. Therefore, once an item is defined in one drawing, it does not require further definition or explanation in the subsequent drawings.

In the description of the disclosure, it also should be noted that, unless otherwise explicitly specified or limited, the terms “arranged”, “mounted”, “connected” and “attached” are to be interpreted broadly, and may be, for example, fixedly connected, detachably connected, or integrally connected; may be mechanically or electrically connected; may be connected directly, or indirectly through an Intermediate medium, or may be an internal communication between two elements. Those of ordinary skill in the art can understand the specific meanings of the above terms in the disclosure in light of specific circumstances.

It should be noted that, without conflicts, the features in the embodiments of the disclosure are able to be combined with each other.

Some embodiments of the disclosure provide a cardiac reconstruction implant, including: an isolation body 100 and a contractile outer mesh 200;
the isolation body 100 and the contractile outer mesh 200 are fixedly connected, and the isolation body 100 is released in a cardiac ventricle of a heart to isolate an ineffective cardiac chamber; and the contractile outer mesh 200 covers a surface of the heart to provide a contractile force to an exterior of the heart.

In some embodiments, the isolation body 100 and the contractile outer mesh 200 are fixedly connected by, but not limited to, integrated forming, adhesive bonding, stapling, welding, or by a connector (such as a cannula, etc.).

In some embodiments, the integrated forming includes, but is not limited to, integrated woven forming, integrated cutting forming, integrated plastic forming, and integrated printing (such as 3D printing) forming, etc.

Not being bound by theory, the term “integrated” as used herein refers to an integrated combination structure composed of one or more structures. These multiple structures are selected from combinations of identical substances or multiple substances within the same material category, or each is selected from combinations of different substances or multiple substances within the same material category.

Not being bound by theory, the term “weave” as used herein refers to an organization or combination of wires or filaments (such as filament materials and wire materials) that are interlaced or hooked together.

In some embodiments, the cardiac reconstruction implant includes: an isolation body 100 and a contractile outer mesh 200;
the isolation body 100 and the contractile outer mesh 200 are integrally formed by weaving, and the isolation body 100 is released in the cardiac ventricle of the heart to isolate the ineffective cardiac chamber; and the contractile outer mesh 200 covers the surface of the heart to provide the contractile force to the exterior of the heart.

In some embodiments, the cardiac reconstruction implant further includes a fixation waist 300;
the isolation body 100 and the contractile outer mesh 200 are fixedly connected by the fixation waist 300; the fixation waist 300 is fixed to a cardiac apex of the heart, so that the contractile outer mesh 200 spreads and covers the surface of the heart with the fixation waist 300 as a center.

In some embodiments, the cardiac reconstruction implant further includes a fixation waist 300;
the isolation body 100 and the contractile outer mesh 200 are integrally woven and connected by the fixation waist 300; the fixation waist 300 is fixed at the cardiac apex of the heart, so that the contractile outer mesh 200 spreads and covers the surface of the heart with the fixation waist 300 as the center. Not being bound by theory, the cardiac apex refers to a perforation position created when the cardiac reconstruction implant passes through the heart.

In some embodiments, the isolation body 100, the fixation waist 300 and the contractile outer mesh 200 are prepared and formed by an elastic material, and the contractile outer mesh 200 covers an exterior of the isolation body 100. In an embodiment, the elastic material includes polymer compounds (such as resins, and elastic polyurethane), metals (such as platinum, palladium, cobalt alloys, nickel-titanium alloys, or cobalt-chromium alloys), etc. In another embodiment, the elastic material includes a filament or a molten material.

In some embodiments, the isolation body 100, the fixation waist 300 and the contractile outer mesh 200 are sequentially formed by weaving an elastic filament, and the contractile outer mesh 200 covers the exterior of the isolation body 100.

In an embodiment, the isolation body, the fixation waist and the contractile outer mesh are also prepared and formed by an elastic material except the elastic filament, and the contractile outer mesh covers the exterior of the isolation body.

In some embodiments, the isolation body 100 includes an expansion segment 101 and an extension segment 102;
the expansion segment 101 is connected to the fixation waist 300 by the extension segment 102, the expansion segment 101 is located at an end, away from the cardiac apex, of the heart, the expansion segment 101 is configured to conform to a ventricle wall, and the extension segment 102 is able to conform to the ventricle wall from the expansion segment 101 to the cardiac apex.

In some embodiments, a diameter of the expansion segment 101 is greater than a diameter of the extension segment 102, and the extension segment 102 fully conforms to a shape of the heart.

In some embodiments, the contractile outer mesh 200 is arcuately expanded along the fixation waist 300 to completely cover the exterior of the heart.

In some embodiments, a distance from an end, away from the fixation waist 300, of the contractile outer mesh 200 to the fixation waist 300 is greater than a distance from the expansion segment 101 to the fixation waist 300, so that the contractile outer mesh 200 is able to gradually cover the exterior of the heart.

In some embodiments, based on the clinical needs of a patient or the shape of the heart of the patient, the distance from the end, away from the fixation waist 300, of the contractile outer mesh 200 to the fixation waist 300 is equal to or less than the distance from the expansion segment 101 to the fixation waist 300.

In some embodiments, the isolation body 100 is a double-layer mesh structure, and an occlusive membrane is also fixed to an outer surface of the double-layer mesh structure of the isolation body 100 to accommodate different patient needs; and the contractile outer mesh 200 is a single-layer or double-layer mesh structure.

In some embodiments, the isolation body 100 is formed as a double-layer woven mesh, and the occlusive membrane is fixed inside the double-layer woven mesh of the isolation body 100; and the contractile outer mesh 200 is formed as a single-layer or double-layer woven mesh.

In some embodiments, the contractile outer mesh 200 is the double-layer mesh structure, and the occlusive membrane is fixed on the contractile outer mesh 200; and the isolation body 100 is a single-layer or double-layer mesh structure, and the occlusive membrane is fixed on the isolation body 100.

In some embodiments, the contractile outer mesh 200 is formed as the double-layer woven mesh, and the occlusive membrane is fixed on the contractile outer mesh 200; and the isolation body 100 is formed as the single-layer or double-layer woven mesh, and the occlusive membrane is fixed on the isolation body 100.

In some embodiments, the contractile outer mesh 200 is a single-layer mesh structure, and the occlusive membrane is fixed on the contractile outer mesh 200; and the isolation body 100 is the single-layer mesh structure, and the occlusive membrane is fixed on the isolation body 100. A delivery system 500 is able to be directly fixedly connected to an end, close to the cardiac apex, of the fixation waist 300 to contract and release the implant.

In some embodiments, the occlusive membrane includes at least one of a polymer membrane, a fabric membrane or a biological tissue membrane.

In some embodiments, the cardiac reconstruction implant further includes a connector 400;
the connector 400 is connected to the fixation waist 300, and the connector 400 is configured to connect to the external delivery system 500 to contract the isolation body 100, the fixation waist 300 and the contractile outer mesh 200 into the external delivery system 500. In some embodiments, the connector 400 is located at an end, close to the cardiac apex, of the contractile outer mesh 200.

In some embodiments, the fixation waist 300 is a single-layer or double-layer mesh structure.

In some embodiments, an end, away from the cardiac apex, of the fixation waist 300 is connected to the extension segment 102, and the end, close to the cardiac apex, of the fixation waist 300 is connected to the contractile outer mesh 200.

In some embodiments, a mesh structure layer of the isolation body 100 in contact with the ventricle wall is provided with at least one fixation anchor 600, and the at least one fixation anchor 600 extends along the mesh structure layer of the isolation body 100 into the ventricle wall.

In some embodiments, a woven layer of the isolation body 100 in contact with the ventricle wall is provided with at least one fixation anchor 600, and the at least one fixation anchor 600 extends along the woven layer of the isolation body 100 into the ventricle wall.

In some embodiments, the fixation waist 300 is provided with a plurality of fixation anchors 600, and the plurality of fixation anchors 600 extend along the fixation waist 300 to the cardiac reconstruction implant and penetrate into a myocardial inner wall of the cardiac apex.

In some embodiments, the isolation body 100 is a goblet-shaped or straw-hat-shaped structure.

In some embodiments, the isolation body 100 is free of a fixation anchor 600, and the expanded isolation body 100 contacts and conforms to the ventricle wall. Not being bound by theory, the isolation body 100 achieves effective isolation and sealing effects.

In some embodiments, a height of the isolation body 100 ranges from 1 mm to 100 mm; and a height of the contractile outer mesh 200 ranges from 1 mm to 100 mm.

In some embodiments, the elastic filament for the isolation body 100, the fixation waist 300 and the contractile outer mesh 200 includes at least one of metal wire, macromolecule filament or ceramic filament.

In an embodiment, the metal wire includes, but is not limited to, elastic platinum wire, elastic palladium wire, cobalt alloy, nickel-titanium alloy, or cobalt-chromium alloy.

For example, the isolation body 100 and the contractile outer mesh 200 are both selectively made of the nickel-titanium alloy. In an embodiment, the isolation body 100 is selectively made of the nickel-titanium alloy, and the contractile outer mesh 200 is selectively made of the elastic palladium wire. In an embodiment, the isolation body 100 is selectively made of a combined elastic filament of an elastic cobalt alloy and the elastic platinum wire (such as through mixed braiding), and the contractile outer mesh 200 is selectively made of the elastic platinum wire.

In some embodiments, the connector 400 is of a riveted type or a flat-end non-riveted type.

In some embodiments, the fixed waist 300 is provided with a fixation anchor 600 on a side close to the cardiac apex of the heart (i.e., an outer side of the fixation waist 300). Not being bound by theory, the fixation anchor 600 on the fixation waist 300 is able to further enhance a fixation effect of the fixation waist 300.

In some embodiments, the process is performed through a cardiac apex intervention or percutaneous intervention, a whole integrated woven structure requires a delivery sheath with a small size, thereby helping to reduce vascular damage and associated complications.

Some other embodiments of the disclosure further provide a cardiac reconstruction method, including: implanting the cardiac reconstruction implant into the heart through the cardiac apex intervention or percutaneous intervention.

The cardiac reconstruction implant provided in the disclosure alleviates the technical problems of the related art, including the increased time and risks of a surgical operation associated with supporting the ventricle wall by a support skeleton and requiring balloon expansion, stress concentration on an instrument skeleton structure that makes it prone to fracture, the unsatisfactory volume reduction effect; and the continued issue of insufficient cardiac contractile force.

The disclosure provides a cardiac reconstruction implant, including: an isolation body 100 and a contractile outer mesh 200; the isolation body 100 and the contractile outer mesh 200 are fixedly connected (such as integrally formed by weaving). The isolation body 100 is released in a cardiac ventricle of a heart, for example, the woven isolation body 100 isolates an ineffective cardiac chamber. The isolation body 100 with a mesh structure (such as a woven structure) has a self-expanding property (which originates from materials with a shape memory functionality or other materials with a certain elasticity). Therefore, balloon expansion is no longer required during an implantation. Moreover, the isolation body 100 with the mesh structure (such as the woven structure) moves with the ventricular motion, and a force applied to the isolation body 100 is dispersed, thereby improving a mechanical performance of the isolation body 100, and reducing a risk of fracture. In an embodiment, the contractile outer mesh 200 covers a surface of the heart. The contractile outer mesh 200 is able to provide a contractile force to an exterior of the heart, i.e., the contractile outer mesh 200 is able to store a certain stress during diastole, and is able to promote heart contraction during systole, thereby achieving the goals of reducing the ventricular volume and ventricular pressure and preventing ventricular dilation, further improving the contractile function for the heart, enhancing the ventricular pumping capacity by isolating the interior of the ventricular wall of the heart and providing a contractile force to the exterior of the heart, and alleviating the technical problems of the related art, including the increased time and risks of a surgical operation associated with supporting the cardiac ventricle wall by a support skeleton and requiring balloon expansion, stress concentration on an instrument skeleton structure that makes it prone to fracture, the unsatisfactory volume reduction effect; and the continued issue of insufficient cardiac contractile force. In addition, the integrated molding of the cardiac reconstruction implant may further improve the stability of the implant fixed to the heart and the longevity and durability of the implant during use.

Furthermore, the fixation waist 300 not only ensures a smooth connection between the isolation body 100 and the contractile outer mesh 200, both inside and outside the heart, but also ensures a stability of the isolation body 100 and the contractile outer mesh 200. Moreover, the waist structure 300 fixed at the cardiac apex is also able to prevent a leakage of cardiac effusion (such as blood leakage).

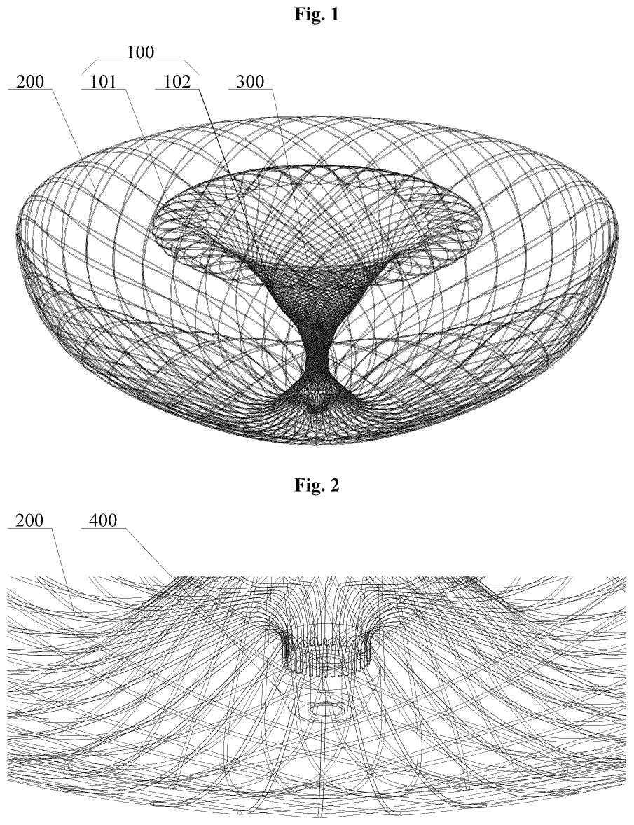

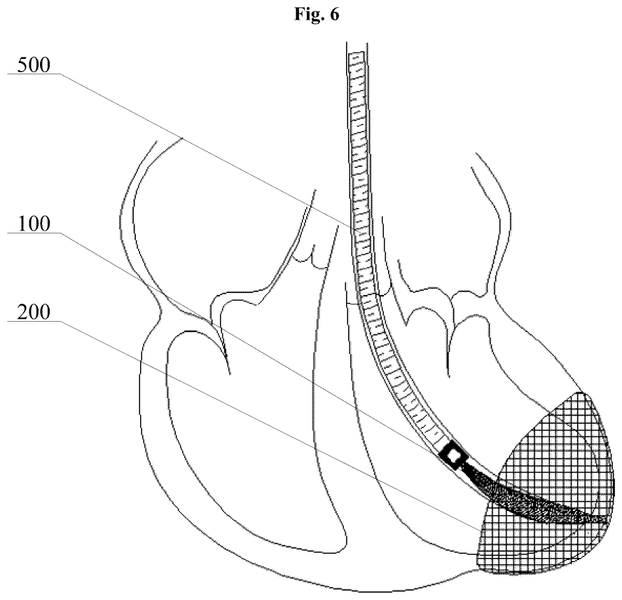

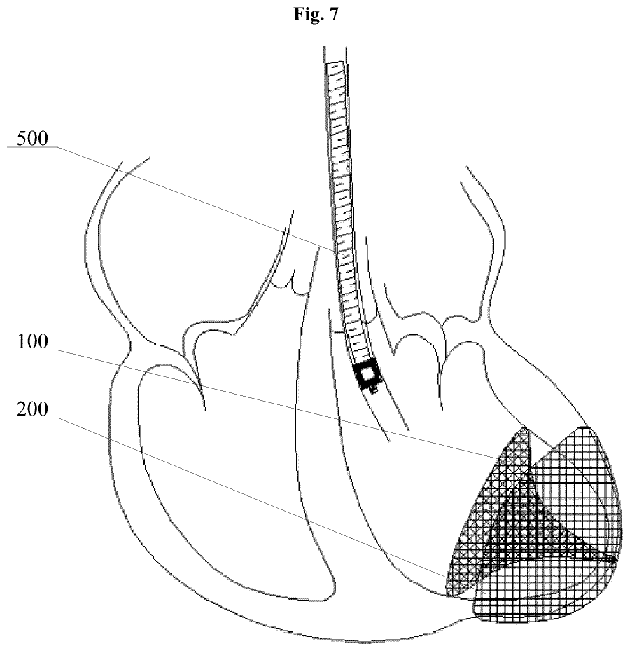

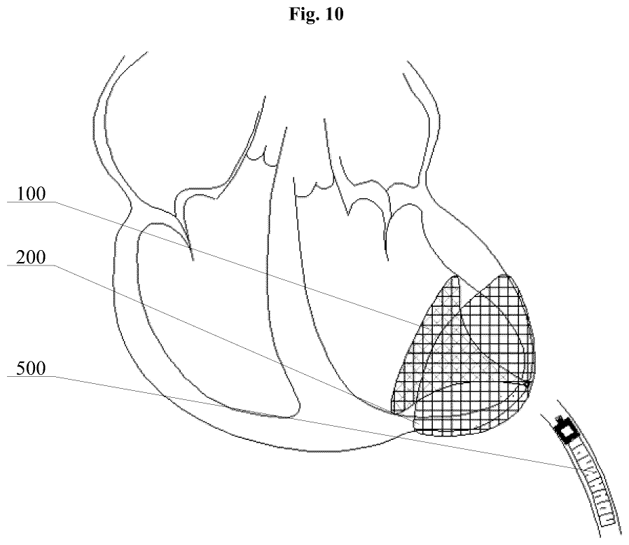

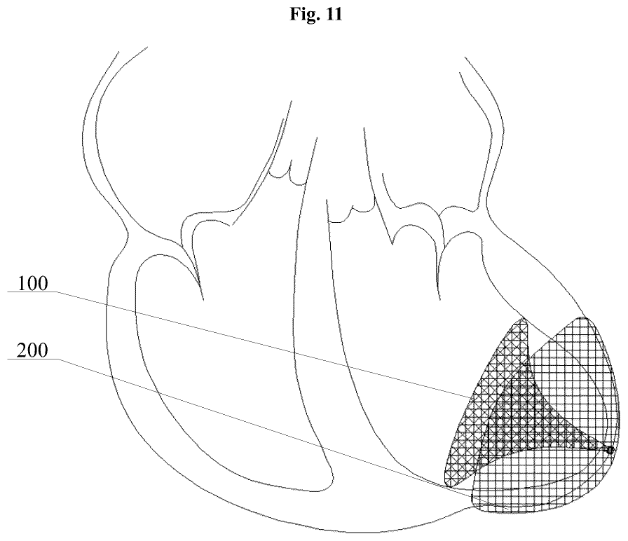

As shown in FIGS. 1-11, some embodiments of the disclosure provide a cardiac reconstruction implant, including: an isolation body 100 and a contractile outer mesh 200; the isolation body 100 and the contractile outer mesh 200 are integrally formed by weaving, and the isolation body 100 is released in a cardiac ventricle of a heart to isolate an ineffective cardiac chamber; and the contractile outer mesh 200 covers a surface of the heart to provide a contractile force to an exterior of the heart.

It should be noted that the present embodiments provide an apparatus for performing cardiac reconstruction and assisting in cardiac contraction, a cardiac reconstruction implant integrating the isolation body 100 and the contractile outer mesh 200 by utilizing a woven structure. In an embodiment, the isolation body 100 and the contractile outer mesh 200 are integrally formed by weaving, and the isolation body 100 is able to isolate an expanded cardiac ventricle into a static cardiac chamber and a dynamic cardiac chamber, thereby isolating ventricular aneurysm and reducing the ventricular volume. In addition, the contractile outer mesh 200 is able to cover an external surface of the heart and assists in cardiac contraction during systole. Since the isolation body 100 and the contractile outer mesh 200 are integrally formed, that is, the isolation and supporting effect of the isolation body 100 on the cardiac ventricle and the contractile force of the contractile outer mesh 200 are able to interact, i.e., the isolation body 100 provides a contractile support force to the contractile outer mesh 200, and the contractile outer mesh 200 ensures the isolation effect of the isolation body 100 on the cardiac chambers. This results in a dual function: isolating the cardiac ventricle and externally providing a contractile force, thereby enhancing the overall design. As shown in FIGS. 1-3, the present embodiment provides a cardiac reconstruction implant, including: an isolation body 100 and a contractile outer mesh 200; the isolation body 100 and the contractile outer mesh 200 are integrally formed by weaving. The isolation body 100 is released into a cardiac ventricle of a heart. An ineffective cardiac chamber is isolated by the woven isolation body 100. The isolation body 100 with a woven structure has a further self-expanding property (the self-expanding property of an elastic material is able to be further improved by braiding, so that the stability of the implant fixed to the heart and the longevity and durability of the implant during use are further improved.) Thus, balloon expansion is no longer required during the implantation. The isolation body 100 with the woven structure moves with the ventricular motion, and a force applied to the isolation body 100 is dispersed, thereby improving a mechanical performance of the isolation body 100, and reducing a risk of fracture. In an embodiment, the contractile outer mesh 200 covers a surface of the heart. The contractile outer mesh 200 is able to provide a contractile force to an exterior of the heart, i.e., the contractile outer mesh 200 is able to store a certain stress during diastole, and is able to promote heart contraction during systole, thereby achieving the goals of reducing the ventricular volume and ventricular pressure and preventing ventricular dilation, further improving the contractile function for the heart, enhancing the ventricular pumping capacity by isolating the interior of the ventricular wall of the heart and providing a contractile force to the exterior of the heart, and alleviating the technical problems of the related art, including the increased time and risks of a surgical operation associated with supporting the cardiac ventricle wall by a support skeleton and requiring balloon expansion, stress concentration on an instrument skeleton structure that makes it prone to fracture, the unsatisfactory volume reduction effect; and the continued issue of insufficient cardiac contractile force.

Based on the above embodiments, in an embodiment, the cardiac reconstruction implant further includes a fixation waist 300; the isolation body 100 and the contractile outer mesh 200 are integrally woven and connected by the fixation waist 300; the fixation waist 300 is fixed to a cardiac apex of the heart, so that the contractile outer mesh 200 spreads and covers the surface of the heart with the fixation waist 300 as a center.

In the embodiment, an end portion of the isolation body 100 is contracted by the fixation waist 300, and the contractile outer mesh 200 is expanded outward from a location of the fixation waist 300. The fixation waist 300 is able to fully pass through the cardiac apex during delivery, i.e., the fixation waist 300 is fixed to the cardiac apex. The contractile outer mesh 200 covers the surface of the heart along the fixation waist 300. As a connecting transition structure of the integrally woven structure, the fixation waist 300 is able to integrate all woven filaments to pass. A smooth connection between the isolation body 100 and the contractile outer mesh 200 inside and outside the heart is ensured by the fixation waist 300. At the same time, the fixation waist 300 is able to ensure a stability of the isolation body 100 and the contractile outer mesh 200. In an embodiment, the isolation body 100, the fixation waist 300 and the contractile outer mesh 200 are sequentially formed by weaving an elastic filament, and the contractile outer mesh 200 covers an exterior of the isolation body 100.

In the embodiment, weaving starting points of the isolation body 100 and the contractile outer mesh 200 are both originate from the fixation waist 300, i.e., all elastic filaments of the isolation body 100 and all elastic filaments of the contractile outer mesh 200 converge at the fixation waist 300. The isolation body 100, the fixation waist 300, and the contractile outer mesh 200 are formed by weaving the elastic filaments. The contractile outer mesh 200 spreads and covers the surface of the heart with the fixation waist 300 as the center. The isolation body 100 also extends with the fixation waist 300 as the center and is arranged on the surface of the ventricle wall. The fixation waist 300 is able to ensure the overall fixation of the instrument at the heart.

In an embodiment, since the isolation body 100, the fixation waist 300 and the contractile outer mesh 200 are sequentially formed by weaving the elastic filament, the properties of the elastic filament allow the isolation body 100 to be implanted without the need for balloon expansion. The isolation body 100 is able to fully conform to the surface of the ventricle wall with ventricular motion. In addition, during the implantation process, the contractile outer mesh 200 expands along the surface of the heart. The contractile outer mesh 200 is able to fully adhere to the surface of the heart. Based on weaving properties of the elastic filament, the contractile outer mesh 200 stores a certain stress as the heart expands during diastole. During systole, the contractile outer mesh 200 is able to apply an acting force to the exterior of the heart to promote cardiac contraction, thereby reducing ventricular volume and wall stress, and preventing ventricular dilation.

In an embodiment, the elastic filament for the isolation body 100, the elastic filament for the fixation waist 300 and the contractile outer mesh 200 includes at least one of metal wire, macromolecule filament or ceramic filament.

In an embodiment, the isolation body 100 is a goblet-shaped or straw-hat-shaped structure. In an embodiment, the isolation body 100 includes an expansion segment 101 and an extension segment 102; the expansion segment 101 is connected to the fixation waist 300 by the extension segment 102, the expansion segment 101 is located at an end, away from the cardiac apex, of the heart, the expansion segment 101 is configured to conform to the ventricle wall, and the extension segment 102 is able to conform to the ventricle wall from the expansion segment 101 to the cardiac apex. In the embodiment, the expansion segment 101 adopts a planar expansion structure, i.e., the isolation body 100 is similar to a shape of an open umbrella. The expansion segment 101 is able to be positioned distal to the cardiac apex, serving as an isolation position between the static cardiac chamber and the dynamic cardiac chamber in the expanded cardiac ventricle. The expansion segment 101 and the extension segment 102 are able to conform to the ventricle wall of the heart, i.e., the myocardium of the heart is able to be isolated from the expansion segment 101 to the extension segment 102. The pressure in the isolated myocardium and the force transmitted to the cardiac apex are both reduced, thus decreasing ventricle wall stress. In addition, apart from a reduced load on the static cardiac chamber, the reduction of a load on the dynamic cardiac chamber also leads to a decrease in normal myocardial pressure, thereby reducing the overall load on the cardiac ventricle.

In an embodiment, a diameter of the expansion segment 101 is greater than a diameter of the extension segment 102, and the extension segment 102 fully conforms to the shape of the heart.

The expansion segment 101 expands the cardiac ventricle in a shape similar to an open umbrella, and due to a gradually narrowing chamber from an interior of the cardiac ventricle to the cardiac apex of the heart, the extension segment 102 is arranged along the expansion segment 101 in a conical shape from the expansion segment 101 to the fixation waist 300. That is, the extension segment 102 is able to function like ribs of the umbrella to gradually contract, thereby allowing the extension segment 102 to conform to the shape of the cardiac ventricle, and ensuring that the static cardiac chamber remains expanded. Meanwhile, the arrangement of the conical extension segment 102 ensures a smooth transition from the end away from the expansion segment 101 to the fixation waist 300, thereby ensuring stable fixation of the fixation waist 300 at the cardiac apex. Furthermore, since the expansion segment 101, the extension segment 102, and the fixation waist 300 are integrally formed by weaving the elastic filament, the stress on the expansion segment 101, the extension segment 102, and the fixation waist 300 is dispersed, avoiding stress concentration in a rod-type skeleton structure, thereby improving the mechanical performance of the entire isolation body 100, reducing the risk of fracture and enhancing the stability of the isolation body 100.

In an embodiment, the contractile outer mesh 200 is arcuately expanded along the fixation waist 300 to completely cover the exterior of the heart. In an embodiment, the contractile outer mesh 200 is expanded in an inwardly curved arcuate shape with the fixation waist 300 as the center in a direction of the isolation body 100 away from the cardiac apex to completely cover the exterior of the heart.

In the embodiment, the contractile outer mesh 200 is in a bowl shape, i.e., the contractile outer mesh 200 expands with the fixation waist 300 as the center. The inner diameter of the contractile outer mesh 200 at the cardiac apex is the smallest. At this point, the contractile outer mesh 200 gradually covers the surface of the heart from the cardiac apex. The contractile outer mesh 200 is also formed by weaving the elastic filament together with the fixation waist 300 and the isolation body 100. As a result, the stress applied by the contractile outer mesh 200 to the heart is also dispersed, avoiding stress concentration at a location of the heart that could cause damage to the surface of the heart. In an embodiment, an inner diameter of the contractile outer mesh 200 changes non-linearly, which means that the inner diameter of the contractile outer mesh 200 is able to be specifically set according to the exterior of the heart, which is not limited here.

In an embodiment, a distance from the end, away from the fixation waist 300, of the contractile outer mesh 200 to the fixation waist 300 is greater than a distance from the expansion segment 101 to the fixation waist 300, so that the contractile outer mesh 200 is able to gradually cover the exterior of the heart.

In the embodiment, since the contractile outer mesh 200, the fixation waist 300, and the isolation body 100 are all installed in an external delivery system 500, a delivery sheath is used for transport. Once the delivery sheath reaches the cardiac apex, the isolation body 100 needs to enter the cardiac ventricle, and then the contractile outer mesh 200 is released to cover the surface of the heart. For easier surgical operation, when the delivery sheath releases the implant apparatus, the isolation body 100 is extended into the cardiac apex first, and then the contractile outer mesh 200 is released, thereby achieving the method of separately delivering the isolation body 100 and the contractile outer mesh 200 to the inside and outside of the heart.

In an embodiment, a height of the isolation body 100 ranges from 1 mm to 100 mm; and a height of the contractile outer mesh 200 ranges from 1 mm to 100 mm.

In an embodiment, the isolation body 100 is formed as a double-layer woven mesh, and an occlusive membrane is fixed inside the double-layer woven mesh of the isolation body 100; and the contractile outer mesh 200 is formed as a single-layer or double-layer woven mesh. In some embodiments, during percutaneous intervention by the instrument, the isolation body 100 should be formed as the double-layer woven mesh, and in this case, the contractile outer mesh 200 is a single-layer woven mesh or the double-layer woven mesh. The isolation body 100 is the double-layer woven mesh, the expansion segment 101 extends to the center of the isolation body 100 and is gathered into the connector 400, and the connector 400 is configured to connect the implant to the delivery system 500. An occlusive membrane should be fixed to the interior or exterior of the isolation body 100 to achieve the function of isolating the effective and ineffective cardiac chambers. When the contractile outer mesh 200 is the single-layer woven mesh, no fixation member is needed. The single-layer woven mesh of the contractile outer mesh 200 is or is not provided with the occlusive membrane. The occlusive membrane on the single-layer woven mesh of the contractile outer mesh 200 is able to be arranged on a surface in contact with the heart or a surface not in contact with the heart. When the contractile outer mesh 200 is the double-layer woven mesh, the double-layer woven mesh of the contractile outer mesh 200 is or is not gathered into a fixation member. The occlusive membrane is or is not arranged on the exterior or interior of the double-layer woven mesh of the contractile outer mesh 200.

The occlusive membrane includes at least one of a polymer membrane, a fabric membrane or a biological tissue membrane.

In an embodiment, the contractile outer mesh 200 is formed as the double-layer woven mesh, and the occlusive membrane is fixed on the contractile outer mesh 200; and the isolation body 100 is formed as the single-layer or double-layer woven mesh, and the occlusive membrane is fixed on the isolation body 100.

In the embodiment, during cardiac apex intervention by the instrument, the contractile outer mesh 200 should be formed as the double-layer woven mesh, and in this case, the isolation body 100 is the single-layer woven mesh or the double-layer woven mesh. Woven filaments of the contractile outer mesh 200 are gathered into the connector 400, and the connector 400 is configured to connect the implant to the delivery system 500. The occlusive membrane should be fixed to the interior or exterior of the isolation body 100. When the isolation body 100 is the single-layer woven mesh, no fixation member is needed. The occlusive membrane should be fixed to a surface of the single-layer woven mesh of the contractile outer mesh 200 to achieve the function of isolating the effective and ineffective cardiac chambers. When the isolation body 100 is the double-layer woven mesh, the double-layer woven mesh of the isolation body 100 is or is not gathered into the fixation component. The occlusive membrane should be fixed to the exterior or interior of the double-layer woven mesh of the isolation body 100, so as to achieve the function of isolating the effective and ineffective cardiac chambers.

In the embodiment, the isolation body 100 is formed as the single-layer woven mesh by the position of the fixation waist 300, or the contractile outer mesh 200 is also formed as the single-layer woven mesh by the position of the fixation waist 300. The single-layer mesh structure provides good elasticity and flexibility, which is able to better protect the cardiac apex. By utilizing the high-density woven mesh structure, a compliance and conformance to the ventricular wall are able to be improved, thereby enhancing the volume reduction effect of the instrument, and also ensuring recyclability.

In an embodiment, the occlusive membrane includes at least one of a polymer membrane, a fabric membrane or a biological tissue membrane.

In the embodiment, since the single-layer woven mesh is in contact with the ventricle wall, the cardiac apex and the surface of the heart, by fixing the occlusive membrane to at least one surface of the single-layer woven mesh, immediate isolation of the cardiac ventricle is able to be achieved, while also ensuring better conformance to the heart surface and the ventricular wall.

In an embodiment, the occlusive membrane is fixed to any surface of the single-layer woven mesh by suturing; meanwhile, occlusive membranes are able to be sutured to both surfaces of the single-layer woven mesh, which is not limited here.

In an embodiment, the isolation body 100, the fixation waist 300 and the contractile outer mesh 200 are formed as the double-layer woven mesh.

In the embodiment, the isolation body 100 is formed as the double-layer woven mesh in a return manner through the position of the fixation waist 300, i.e., a portion, away from the fixation waist 300, of the isolation body 100 is woven in an arcuate return manner, or the contractile outer mesh 200 is also formed as the double-layer woven mesh through the position of the fixation waist 300, i.e., a portion, away from the fixation waist 300, (along a direction, away from the cardiac apex, of the isolation body 100) of the contractile outer mesh 200 is woven in an arcuate return manner. The double-layer woven mesh is able to better ensure the overall stability of the instrument. Meanwhile, the high-density woven mesh structure is able to improve the compliance and conformance of the ventricular wall, thereby enhancing the volume reduction effect of the instrument.

In the embodiment, the occlusive membrane is fixed between layers of the double-layer woven mesh, so that the double-layer woven mesh structure is able to be in contact with and conform to the surface of the heart and the ventricular wall, and immediate isolation of the cardiac ventricle is also able to be achieved through the flow-blocking membrane.

In an embodiment, the occlusive membrane is able to be fixed between layers of the double-layer woven mesh by suturing.

As shown in FIG. 2, in an embodiment, the cardiac reconstruction implant further includes a connector 400. The connector 400 is connected to an end, away from the isolation body 100, of the fixation waist 300, and the connector 400 is configured to connect to the external delivery system 500 so as to contract the isolation body 100, the fixation waist 300 and the contractile outer mesh 200 into the external delivery system 500.

In the embodiment, the connector 400 is fixedly connected to the fixation waist 300, wherein a diameter range of the connector 400 is a diameter range of the fixed waist 300 or less than the diameter of the fixation waist 300. In some embodiments, the connector 400 is located at an end, close to the cardiac apex, of the contractile outer mesh 200, and the connector 400 is configured to connect the cardiac reconstruction implant with the external delivery system 500, so that the cardiac reconstruction implant is delivered to a target position by the delivery system 500. In some embodiments, when the contractile outer mesh 200 and the isolation body 100 are fully contracted, the fixation waist 300 serves as a contraction collection position. The fixation waist 300 is connected to the external delivery system 500 by the connector 400. The external delivery system 500 is able to be connected to the implant by the connector 400 for delivery. In an embodiment, the connector 400 is of a riveted type or a flat-end non-riveted type, where the connector 400 should only ensure the connection between the implant and the external delivery system 500, and the structure of the connector 400 is not limited here.

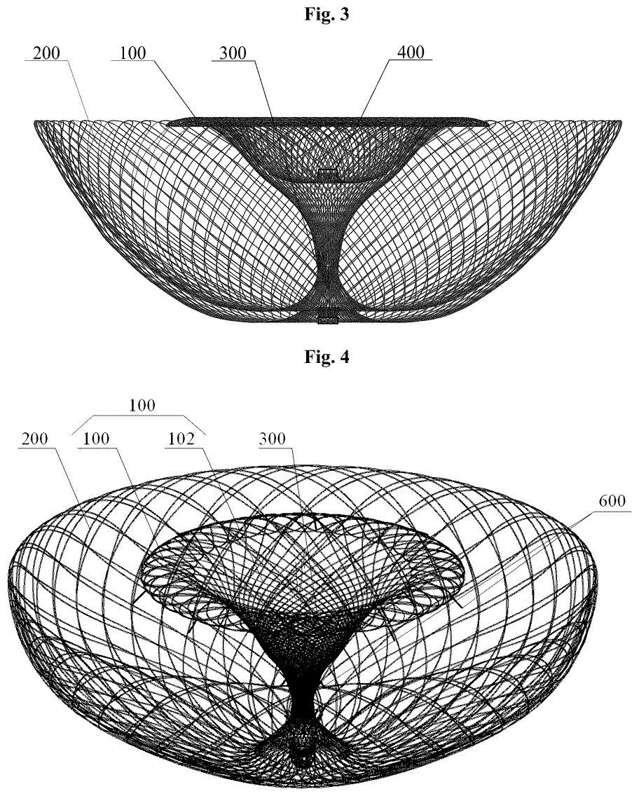

As shown in FIG. 4, in an embodiment, a woven layer of the isolation body 100 in contact with the ventricle wall is provided with a plurality of fixation anchors 600, and the plurality of fixation anchors 600 extend into the ventricle wall along the woven layer of the isolation body 100.

In the embodiment, the plurality of fixation anchors 600 are positioned at an outer periphery of the expansion segment 101. When the isolation body 100 is placed, the plurality of fixation anchors 600 are able to penetrate into the myocardium of the ventricular wall. The overall anchoring of the isolation body 100 is able to be ensured by the plurality of fixation anchors 600, thereby improving the overall stability of the placement of the isolation body 100.

In an embodiment, the plurality of fixation anchors 600 are provided. The plurality of fixation anchors 600 are inclined in multiple directions. In an embodiment, during systole/diastole, in addition to the expansion and contraction of the cardiac ventricle, the myocardial spiral motion also occurs. The myocardial motion amplitude and direction at the fixation point of each fixation anchor 600 differ to some extent. By designing the direction of the fixation anchors 600 to be multi-directional, the implantation of the instrument is able to be improved. When the plurality of fixation anchors 600 are anchored to the myocardium of the ventricular wall, they penetrate into the ventricular wall along a motion direction of the heart, further improving the stability of anchoring and the anchoring stability of the isolation body 100.

In an embodiment, the isolation body 100 is or is not provided with a fixation anchor 600. In an embodiment, the isolation body 100 is free of the fixation anchor 600, as long as the expanded isolation body 100 is in contact with and conforms to the ventricular wall.

As shown in FIG. 4, the fixation waist 300 is further provided with a plurality of fixation anchors 600, and the plurality of fixation anchors 600 extend along the fixation waist 300 to the cardiac reconstruction implant and penetrate into the myocardial inner wall of the cardiac apex, thereby further enhancing the stability of the cardiac reconstruction implant fixed to the heart. In an embodiment, the implantation of the implant is performed through cardiac apex intervention or percutaneous intervention, the whole woven structure requires a delivery sheath of a small size, thereby helping to reduce vascular damage and associated complications.

It should be noted that the present embodiments provides an apparatus for performing cardiac reconstruction and assisting in cardiac contraction, a cardiac reconstruction implant integrating the isolation body 100 and the contractile outer mesh 200 by utilizing a woven structure. In an embodiment, the isolation body 100 and the contractile outer mesh 200 are integrally formed by weaving, and the isolation body 100 is able to isolate an expanded cardiac ventricle into a static cardiac chamber and a dynamic cardiac chamber, thereby isolating ventricular aneurysm and reducing the ventricular volume. In addition, the contractile outer mesh 200 is able to cover the external surface of the heart and assists in cardiac contraction during systole. Since the isolation body 100 and the contractile outer mesh 200 are integrally formed, that is, the isolation and supporting effect of the isolation body 100 on the cardiac ventricle and the contractile force of the contractile outer mesh 200 are able to interact, i.e., the isolation body 100 provides a contractile support force to the contractile outer mesh 200, and the contractile outer mesh 200 ensures the isolation effect of the isolation body 100 on the cardiac chambers. This results in a dual function: isolating the cardiac ventricle and externally providing a contractile force, thereby enhancing the overall design.

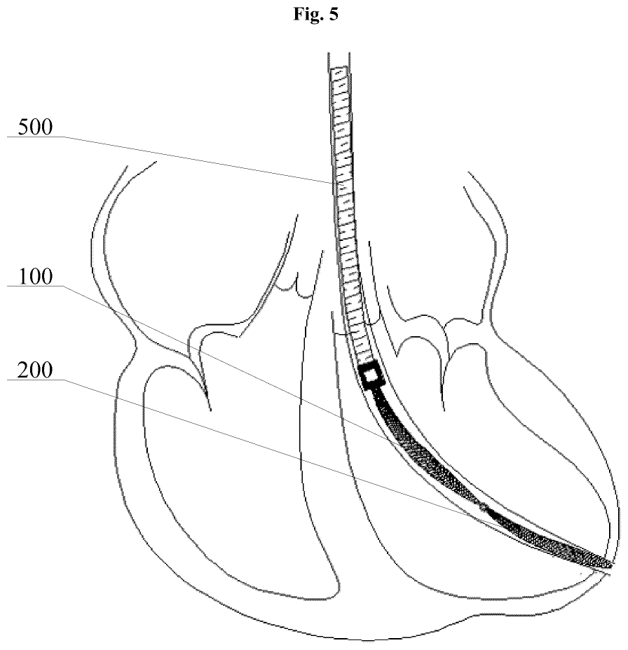

Referring to FIGS. 5-7, through percutaneous intervention, by puncturing a blood vessel on a body surface, during the preliminary process, the intermediate process and the completed process of releasing the cardiac reconstruction implant into the left ventricle and covering the external of the heart under the guidance of the delivery system 500, the cardiac reconstruction implant releases the contractile outer mesh 200 to the exterior of the heart first under the guidance of the delivery system 500, the fixation waist 300 is fixed to the cardiac apex, the contractile outer mesh 200 is arcuately expanded along the fixation waist 300 with the fixation waist 300 as the center, and fully covers the external surface of the heart along the exterior of the heart, so that the contractile outer mesh 200 assists in the contraction during systole; and subsequently, the isolation body 100 is released into the cardiac ventricle to isolate the expanded cardiac ventricle into a static cardiac chamber and a dynamic cardiac chamber.

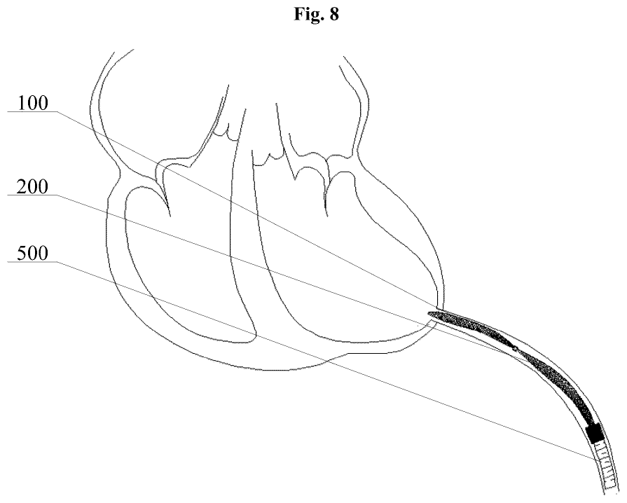

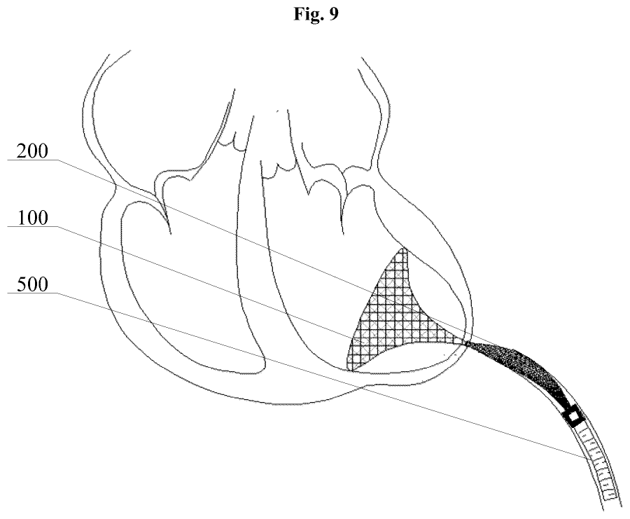

Referring to FIGS. 8-11, through cardiac apex intervention, during the preliminary process, the intermediate process and the completed process of releasing the cardiac reconstruction implant into the left ventricle and covering the external of the heart under the traction of the delivery system 500, the cardiac reconstruction implant releases the isolation body 100 into the cardiac ventricle first under the traction of the delivery system 500 to isolate the expanded cardiac ventricle into a static cardiac chamber and a dynamic cardiac chamber; subsequently, the fixation waist 300 is fixed to the cardiac apex; and finally, the contractile outer mesh 200 is arcuately expanded along the fixation waist 300 with the fixation waist 300 as the center, and fully covers the outer surface of the heart along the exterior of the heart to assist in the contraction during systole, thereby further improving the contractile function for the heart.

Finally, it should be noted that the above embodiments are only configured to illustrate the technical solutions of the disclosure, not to limit the disclosure; although the disclosure has been illustrated in detail by referring to the aforementioned embodiments, those of ordinary skill in the art should understand that: they can still make modifications to the technical solution recorded in each aforementioned embodiment, or make equivalent replacements to part or all of the technical features thereof, but these modifications or replacements do not make the nature of the corresponding technical solution departing from the scope of the technical solution of each embodiment of the disclosure.

### INDUSTRIAL APPLICABILITY

The cardiac reconstruction implant disclosed herein features a special woven structure that eliminates the need for balloon expansion during implantation. The force applied to the isolation body is dispersed, which enhances the mechanical performance of the isolation body. The contractile outer mesh covers the surface of the heart, thereby achieving the goals of reducing the ventricular volume and ventricular pressure and preventing ventricular dilation, enhancing the ventricular pumping capacity, and alleviating the technical problems of the related art, including the time and risks of a surgical operation, stress concentration on an instrument skeleton structure that makes it prone to fracture, the unsatisfactory volume reduction effect; and the continued issue of insufficient cardiac contractile force. Therefore, the cardiac reconstruction implant disclosed herein offers excellent industrial applicability and has a broad market prospect.

1. A cardiac reconstruction implant, comprising: an isolation body and a contractile outer mesh;
wherein the isolation body and the contractile outer mesh are integrally formed by weaving, and the isolation body is released in a cardiac ventricle of a heart to isolate an ineffective cardiac chamber; and the contractile outer mesh covers a surface of the heart to provide a contractile force to an exterior of the heart.

2. The cardiac reconstruction implant according to claim 1, wherein the cardiac reconstruction implant further comprises a fixation waist;
the isolation body and the contractile outer mesh are integrally woven and connected by the fixation waist; the fixation waist is fixed at a cardiac apex of the heart, so that the contractile outer mesh spreads and covers the surface of the heart with the fixation waist as a center.

3. The cardiac reconstruction implant according to claim 2, wherein the isolation body, the fixation waist and the contractile outer mesh are sequentially formed by weaving an elastic filament, and the contractile outer mesh covers an exterior of the isolation body.

4. The cardiac reconstruction implant according to claim 2, wherein the isolation body comprises an expansion segment and an extension segment;
the expansion segment is connected to the fixation waist by the extension segment, the expansion segment is located at an end, away from the cardiac apex, of the heart, the expansion segment is configured to conform to a ventricle wall, and the extension segment is able to conform to the ventricle wall from the expansion segment to the cardiac apex.

5. (canceled)

6. The cardiac reconstruction implant according to claim 1, wherein the contractile outer mesh is arcuately expanded along the fixation waist to completely cover the exterior of the heart.

7. The cardiac reconstruction implant according to claim 3, wherein
the isolation body is formed as a double-layer woven mesh, and an occlusive membrane is fixed inside the double-layer woven mesh of the isolation body, and the contractile outer mesh is formed as a single-layer or double-layer woven mesh; or
the isolation body is formed as the double-layer woven mesh, and the occlusive membrane is fixed on a surface of the double-layer woven mesh of the isolation body, and the contractile outer mesh is formed as the single-layer or double-layer woven mesh; or
the isolation body is formed as the single-layer woven mesh, and the occlusive membrane is fixed on a surface of the single-layer woven mesh or an inner cavity of the isolation body, and the contractile outer mesh is formed as the single-layer woven mesh.

8. The cardiac reconstruction implant according to claim 3, wherein the contractile outer mesh is formed as a double-layer woven mesh, and an occlusive membrane is fixed on the contractile outer mesh; and
the isolation body is formed as a single-layer or double-layer woven mesh, and the occlusive membrane is fixed on the isolation body.

9. A cardiac reconstruction implant, comprising: an isolation body and a contractile outer mesh;
wherein the isolation body and the contractile outer mesh are fixedly connected, and the isolation body is released in a cardiac ventricle to isolate an ineffective cardiac chamber of a heart; and the contractile outer mesh covers a surface of the heart to provide a contractile force to an exterior of the heart.

10. The cardiac reconstruction implant according to claim 9, wherein the cardiac reconstruction implant further comprises a fixation waist;
the isolation body and the contractile outer mesh are fixedly connected by the fixation waist; the fixation waist is fixed to a cardiac apex of the heart, so that the contractile outer mesh spreads and covers the surface of the heart with the fixation waist as a center.

11. The cardiac reconstruction implant according to claim 10, wherein the isolation body, the fixation waist and the contractile outer mesh are prepared and formed by an elastic material, and the contractile outer mesh covers an exterior of the isolation body.

12. The cardiac reconstruction implant according to claim 11, wherein the isolation body comprises an expansion segment and an extension segment;
the expansion segment is connected to the fixation waist by the extension segment, the expansion segment is located at an end, away from the cardiac apex, of the heart, the expansion segment is configured to conform to a ventricle wall, and the extension segment is able to conform to the ventricle wall from the expansion segment to the cardiac apex.

13. The cardiac reconstruction implant according to claim 12, wherein a diameter of the expansion segment is greater than a diameter of the extension segment, and the extension segment fully conforms to a shape of the heart.

14. The cardiac reconstruction implant according to claim 13, wherein the contractile outer mesh is arcuately expanded along the fixation waist to completely cover the exterior of the heart.

15. The cardiac reconstruction implant according to claim 11, wherein the isolation body is a double-layer mesh structure, and an occlusive membrane is fixed to the double-layer mesh structure of the isolation body; and
the contractile outer mesh is a single-layer or double-layer mesh structure.

16. The cardiac reconstruction implant according to claim 11, wherein
the contractile outer mesh is a double-layer mesh structure, and an occlusive membrane is fixed on the contractile outer mesh; or
the isolation body is a single-layer or double-layer mesh structure, and the occlusive membrane is fixed on the isolation body; or
the contractile outer mesh is a single-layer mesh structure, and the occlusive membrane is fixed on the contractile outer mesh; and the isolation body is a single-layer mesh structure, and the occlusive membrane is fixed on the isolation body.

17. The cardiac reconstruction implant according to claim 2, wherein the cardiac reconstruction implant further comprises a connector;
the connector is connected to the fixation waist, and the connector is configured to connect to an external delivery system to contract the isolation body, the fixation waist and the contractile outer mesh into the external delivery system.

18. The cardiac reconstruction implant according to claim 3, wherein
a woven layer of the isolation body in contact with a ventricle wall is provided with at least one fixation anchor, and the fixation anchor extends into the ventricle wall along the woven layer of the isolation body; or
the fixation waist is provided with one or more fixation anchors, and the one or more fixation anchors extend along the fixation waist to the cardiac reconstruction implant and penetrate into a myocardial inner wall of the cardiac apex.

19. The cardiac reconstruction implant according to claim 9, wherein
a mesh structure layer of the isolation body in contact with a ventricle wall is provided with one or more fixation anchors, and the one or more fixation anchors extend along the mesh structure layer of the isolation body into the ventricle wall; or
the fixation waist is provided with one or more fixation anchors, and the one or more fixation anchors extend along the fixation waist to the cardiac reconstruction implant and penetrate into a myocardial inner wall of the cardiac apex.

20. A cardiac reconstruction method, comprising: implanting the cardiac reconstruction implant according to claim 1 into a heart through a cardiac apex intervention or percutaneous intervention.

21. The cardiac reconstruction implant according to claim 4, wherein the isolation body is a goblet-shaped or straw-hat-shaped structure, and the extension segment is arranged along the expansion segment in a conical shape from the expansion segment to the fixation waist.
